(19) United States
(12) Patent Application Publication
(10) Pub. No.: US 2006/0234907 A1
(43) Pub. Date: Oct. 19, 2006

(54) Albumin solution and process for the production thereof

(76) Inventors: Werner Gehringer, Wien (AT); Katharina Pock, Streifing (AT); Jürgen Römisch, Gramatneusiedl (AT); Tor-Einar Svae, Mödling (AT); Christoph Kannicht, Berlin (DE)

Correspondence Address: STERNE, KESSLER, GOLDSTEIN & FOX PLLC, 1100 NEW YORK AVENUE, N.W., WASHINGTON, DC, 20005, US

(21) Appl. No.: 10/544,833

(22) PCT Filed: Feb. 13, 2004
(86) PCT No.: PCT/EP04/01397

§ 371 Date: May 23, 2006

(51) Int. Cl. C07K 14/765

(52) U.S. Cl. 514/2; 530/364

(57) ABSTRACT

The invention relates to a therapeutically usable virus-inactivated albumin, and to a process for the preparation of a therapeutically usable virus-inactivated albumin, characterized by the combination of the following steps: (a) subjecting a first aqueous albumin solution to a treatment for virus inactivation by the SD method by contacting it with SD reagents at a temperature of below 45° C.; (b) removing, at least substantially, the SD reagents by oil extraction followed by hydrophobic interaction chromatography, wherein a hydrophobic matrix, especially a matrix to which hydrophobic groups may optionally be bound, is used for said chromatography, with the proviso that said groups are aliphatic groups with more than 24 carbon atoms, to obtain a second albumin solution to which
(c) optionally one or more stabilizers selected from the group of sugars, amino acids and sugar alcohols are added, with the proviso that no indole stabilizer and no C₆-C₁₀ fatty acid is employed as said stabilizer, whereupon (d) said second albumin solution to which a stabilizer has optionally been added is subjected to final packaging and sterile filtration and optionally filled into final containers.

The invention relates to a therapeutically usable virus-inactivated albumin, and to a process for the preparation thereof.

Albumin is the plasma protein with the highest proportion in blood plasma. Albumin can bind many endogenous and exogenous substances to its molecule. This binding capacity is also the basis of one of its main functions: the transport of the substances bound to albumin.

Due to this binding capacity, albumin is also an important depot for a wide variety of compounds, such as long-chain fatty acids, bilirubin, tryptophan, thyroxine or metal ions. Administered pharmaceutically active substances, such as warfarin, digitoxin or naproxen, are also bound to albumin and transported.

However, in this connection, it is critical to know that only the free fraction of the respective pharmaceutically active substance, i.e., that which is not bound to albumin, is the one that displays the pharmacological action. A reduction of the portion bound to albumin increases the free fraction and thus the pharmacological activity.

All commercially available albumin preparations are prepared by means of a modified Cohn fractionation, a method which usually consists of several fractionation steps. Pasteurization (10 hours at 60° C.) of the albumin concentrate has been employed as the virus-inactivation step for decades. To avoid the denaturing of albumin during this step, stabilizers are employed. According to the European Pharmacopoeia, sodium caprylate (sodium octanoate) or N-acetyltryptophan or a combination of both is used as a stabilizer.

To obtain virus-inactivated factor VIII preparations or other plasma proteins, the so-called SD method is employed, as described, for example, in EP-A-0 131 740. This laid-open specification is included herein by reference.

From his own studies, Applicant knows that the binding capacity of commercially available albumin is considerably reduced as compared to natural albumin. This is explained by the fact that the stabilizers used in the pasteurization are bound by albumin and thus occupy important transport sites, whereby the binding capacity is decreased. This means that patients which obtain such albumin preparations are exposed to a significantly increased concentration of free active substance, i.e., one which is not bound to albumin, when pharmaceutically active substances are administered, which naturally means an increased risk of exceeding pharmacological effects and side effects for the patient.

It is the object of the present invention to provide an albumin preparation which does not have this disadvantage.

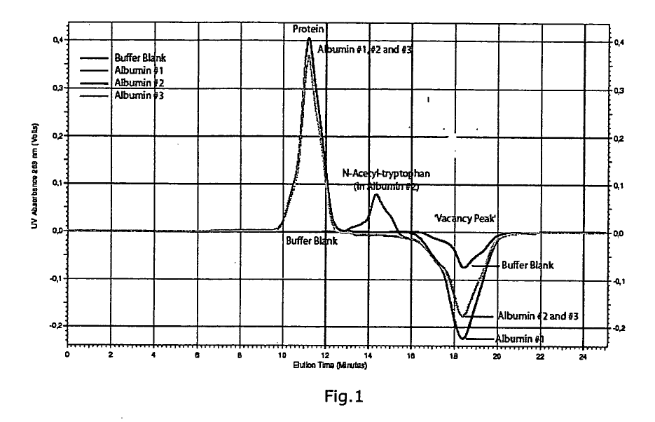

FIG. 1 shows the ultraviolet absorption behavior of albumins from different sources as a function of the elution time during the chromatographic separation.

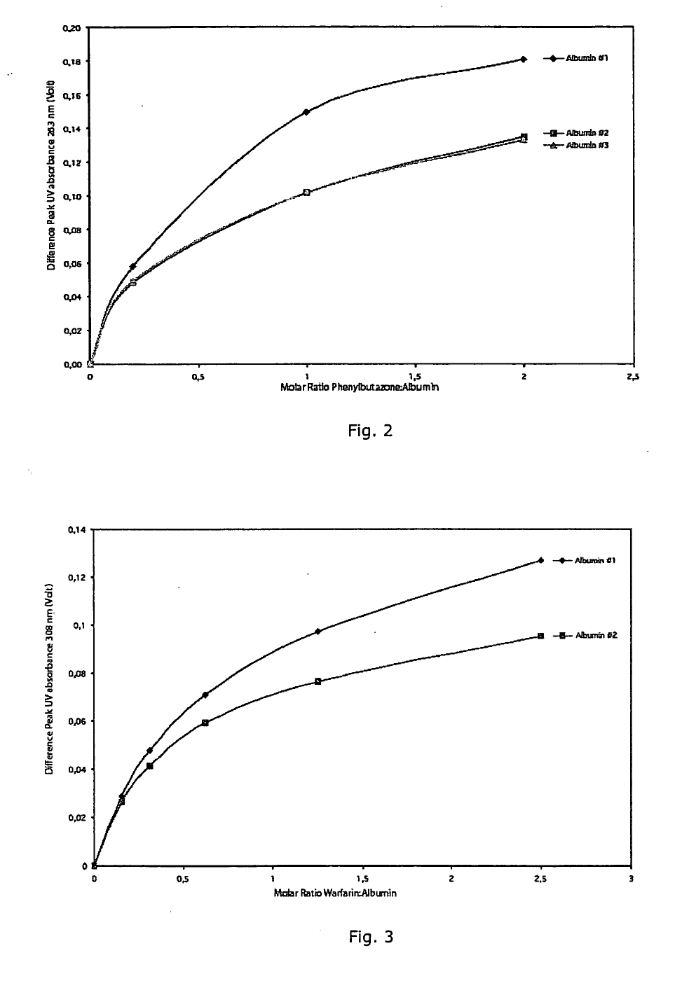

FIG. 2 illustrates the binding behavior of albumins from different sources in the presence of different concentrations of phenylbutazone.

FIG. 3 illustrates the binding behavior of albumins from different sources in the presence of different concentrations of warfarin.

This object is achieved by a therapeutically usable virus-inactivated albumin having an increased binding capacity for substances as compared to albumin virus-in-activated by pasteurization. In particular, the albumin according to the invention has a binding capacity which is increased by at least 10% over that of albumin virus-inactivated by pasteurization, typically a binding capacity which is increased by from 20 to 500%, especially one which is increased by from 100 to 500%. In singular cases, even higher values are possible, depending on the substance to be bound.

The substances are especially those which are bound and/or transported by native albumin, particularly including low-molecular weight active substances. In particular, the low-molecular weight active substances are organic or inorganic substances, nucleic acids, polypeptides, which typically have a molecular weight of <10 000 Da.

For the therapeutical uses mentioned above, the albumin according to the invention can be in the form of a liquid solution or in a solid state, especially in a lyophilized form.

The albumin according to the invention can also be obtained by a process which is characterized by the combination of the following steps:
(a) subjecting a first aqueous albumin solution to a treatment for virus inactivation by the SD method by contacting it with SD reagents at a temperature of below 45° C.;
(b) removing, at least substantially, the SD reagents by oil extraction followed by hydrophobic interaction chromatography, wherein a hydrophobic matrix, especially a matrix to which hydrophobic groups may optionally be bound, is used for said chromatography, with the proviso that said groups are aliphatic groups with more than 24 carbon atoms, to obtain a second albumin solution to which
(c) optionally one or more stabilizers selected from the group of sugars, amino acids and sugar alcohols are added, with the proviso that no indole stabilizer and no C₆-C₁₀ fatty acid is employed as said stabilizer, whereupon
(d) said second albumin solution to which a stabilizer has optionally been added is subjected to final packaging and sterile filtration and optionally filled into final containers.

The term “indole stabilizer” shall comprise all stabilizers which have an indole skeleton, such as N-acetyltryptophan.

The SD (=solvent/detergent) method for the inactivation of viruses has been known from EP-A-0 131 740. This specification also mentioned albumin, among other proteins.

It is true, from EP-A-0 366 946, it is known that the SD reagents can be removed with vegetable oils, for example, soybean oil, followed by hydrophobic interaction chromatography. Thus, as far as it overlaps with the process according to EP-A-0 366 946, the process according to claim 8 is to be considered as an analogous process for the preparation of the albumin according to the invention in one aspect. However, for chromatography, the above patent preferably proposes a matrix, for example, a silica matrix, to which hydrophobic side chains, i.e., branched or unbranched C₆-C₂₄ alkyl chains, are bound.

Surprisingly, it has been found that the use of a hydrophobic matrix instead of a matrix which bears C18 alkyl chains, for example, as hydrophobic side chains results in a higher binding capacity for the adsorption of detergents. Accordingly, no further hydrophobic groups need to be bound to the matrix employed according to the invention. Therefore, the invention also relates to a process in which such a matrix is used.

The virus inactivation is advantageously effected at a temperature within a range of from 25 to 40° C.

In a preferred embodiment of the process according to the invention, the virus inactivation is effected during a period of time within a range of from 4 to 6 hours.

Glycine is very suitable as a stabilizer.

Castor oil is very suitable for oil extraction.

It has been found of particular advantage to the purification effect if a polystyrene-divinylbenzene polymer or a methacrylate-based polymer is used as said hydrophobic matrix.

The hydrophobic matrices employed according to the invention can bear branched or linear aliphatic groups with more than 24 carbon atoms.

Depending on the starting material employed, a step for depletion of the so-called prekallikrein activator (PKA) activity may be required. PKA is known to cause the drop of blood pressure after the administration of PKA-containing preparations by releasing the vaso-active substance bradykinin from high molecular weight kininogen (HMWK).

PKA is usually inactivated during the pasteurization of protein preparations. Since a heat treatment, by which PKA is at least partially inactivated as known from former experience, is disadvantageous to the albumin prepared according to the invention for the reasons mentioned above, PKA can be removed by special measures, if required. These include incubation with active charcoal followed by filtration, preferably with deep filters, or direct filtration through filters containing active charcoal.

Further, ion-exchangers, such as cation or anion exchangers, are very suitable for removing PKA. This may be effected by contacting the albumin-containing solution with the matrix in columns, or by batch processes known to the skilled person. Alternatively, dextran sulfate or heparin matrices may be employed for the reduction of PKA.

In the albumin-containing solution obtained, PKA is reduced, and no longer detectable in the optimum case. According to the current state of the art, PKA is identical with the activated (coagulation) factor XII (FXIIa), which is generated from its pro-enzyme form (FXII). This can occur on surfaces by autocatalysis or by enzymatic action, for example, of kallikrein. Accordingly, depletion of FXII (the pro-enzyme), being a precursor of PKA, is also recommendable, but not necessarily required. However, to prevent the renewed generation of PKA from the pro-enzyme form, the latter may also be removed by ion-exchange chromatography. The depletion of the FXII may optionally be performed in order to enable the long-term storage of albumin in a liquid state. This is also important after the thawing of an albumin solution which may have been stored in a frozen state. Accordingly, the

albumin solution may be deep-frozen after being filled into the final containers, but it may also be cooled in a liquid or freeze-dried state and stored at a temperature of up to 40° C.

Thus, to remove PKA or PKA-precursor substances, any prekallikrein activator (PKA) activity which may be present before or after steps (a), (b) or (c) can be removed in a per se known manner, in particular wherein the albumin solution is

A) contacted with active charcoal, followed by removing the active charcoal from the albumin solution; or

B) subjected to ion-exchange chromatography.

Step (A) is effected at an albumin concentration of from 1 to 25% by weight, especially from 5 to 10% by weight.

Step (B) is performed, in particular, at an albumin concentration of from 5 to 10% by weight.

In a further embodiment of the process according to the invention, the ion-exchanger is an anion-exchanger, and the albumin solution is buffered with sodium acetate within a range of from 100 to 150 mmol/l, and the pH is within a range of from 5.0 to 6.0, especially <5.5.

Further, a process is described which is characterized in that said ion-exchanger is a cation exchanger, and the albumin solution is buffered with sodium acetate within a range of from 20 to 30 mmol/l, and the pH value is within a range of from 4.8 to 6.0, especially within a range of from 4.8 to 5.2.

The invention further relates to an albumin solution which can be obtained by the process according to the invention. This process can be applied to albumin solutions obtained from different sources, for example, from blood plasma or serum, from albumin-containing fractions of plasma fractioning, from albumin recovered from the culture supernatant after recombinant preparation, or from transgenically prepared albumin, or from a medium containing the albumin, such as milk.

A preferred embodiment of the invention is further described by means of the following Example.

### EXAMPLE

To 1000 g of an aqueous albumin solution obtained by the Cohn method (after diafiltration/ultrafiltration) and having a protein content of about 23% are added Triton X-100 and tri-n-butylphosphate (TNBP) up to a concentration of 1% each. Subsequently, the albumin solution is stirred at 30° C. for 4 hours.

To remove the SD reagents, castor oil is first added with stirring up to a concentration of 5% while the solution is brought to a temperature within a range of from 20 to 25° C. Thereafter, the mixture is stirred for 30 minutes. After the stirring, the mixture is allowed to stand for 60 minutes to form a heavy aqueous and a light phase. The heavy phase is separated off and filtered through a filter having membranes with a pore size of <1 μm and <0.45 μm. The light phase (oil phase) contains the TNBP and is discarded.

To separate off the Triton X-100, the filtered solution is passed through a solid-phase extraction column. A polystyrene-divinylbenzene polymer (Amberchrome CG 161) without hydrophobic side chains is used as the hydrophobic matrix. Water for injection is used for purging the column, which process is monitored by measuring the ultraviolet absorption at 280 nm. After use, the column is regenerated.

The following can be added as stabilizers: glycine, glutamate, arginine, maltose, sorbitol or mixtures of these substances.

The solution obtained is brought to pH 7.0, and the protein content is adjusted to 200 g/l, and the sodium content to 80 mmol/l Na⁺. Then, the solution is subjected to sterile filtration through a membrane filter having a pore size of ≦0.2 μm.

The sterile-filtered solution is filled into sterile and pyrogen-free PVC bags under aseptic conditions, and the bags are labeled.

The labeled bags are deep-frozen at a temperature of <−60° C. so that the temperature within the bags reaches <−30° C. At this temperature (<−30° C.), the bags are stored.

Prekallikrein Depletion

When PKA is to be depleted, the following procedure variants can be used:
a) An albumin solution having a protein concentration of from 1 to 25% by weight, especially from 5 to 10% by weight, is stirred for one hour with 3-10% by weight, especially 5% by weight, of active charcoal at pH=5.

Subsequently, the active charcoal is filtered off.
b) An albumin solution having a protein concentration of from 5 to 10% by weight is subjected to ion-exchange chromatography (DEAE Sepharose, Q Sepharose) at pH 5-6, especially <5.5, in a system buffered with 100-150 mM sodium acetate. Due to the high ion strength, a PKA-free albumin solution is obtained in the filtrate.
c) An albumin solution having a protein concentration of from 5 to 10% by weight is subjected to ion-exchange chromatography (SP Toyopearl, CM Sepharose) at pH 5-6, preferably 4.8-5.2, in a system buffered with 20-30 mmol/l sodium acetate. A PKA-free albumin solution is obtained in the filtrate.
   Final Formulation

The solutions obtained are brought to pH=7.0 each, and the protein content is adjusted to 200 g/l, and the sodium content to 80 mmol/l Na⁺. Then, the solution is subjected to sterile filtration through a membrane filter having a pore size of <0.2 μm.

The sterile-filtered solutions are filled into sterile and pyrogen-free PVC bags under aseptic conditions, and the bags are labeled.

The labeled bags are deep-frozen at a temperature of <−60° C. so that the temperature within the bags reaches <−30° C. At this temperature (≦−30° C.), the bags are stored.

Measurement of the Binding of Substances to Different Albumin Preparations

A direct method for determining the binding properties of substances to albumin is the size-exclusion chromatography (SEC) according to Hummel and Dreyer (Biochim Biophys Acta 1962; 63: 530-532).

Thus, an SEC column is equilibrated with a buffer solution containing the binding ligand (e.g., phenylbutazone or warfarin). The absorption in the ultraviolet region is continuously monitored. The protein is applied to the column and eluted in the equilibration buffer. Bound ligand becomes eluted together with the albumin, while the non-bound ligand, which is smaller in most cases, becomes eluted correspondingly later. The absorption of the bound ligand mostly interferes with the absorption of the albumin and possible accompanying substances, such as stabilizers. The later eluting “negative” or so-called “vacancy” peak is caused by the depletion of the ligand in the subsequent buffer, which occupies the larger a surface area, the more binding to the previously eluted albumin occurred. Koizumi et al. (Biomed Chromatogr 1998; 12: 203-210) used this method in a slightly modified form to examine the binding capacities of substances to albumin or their affinities, for example, by adding increasing amounts of the ligand to constant concentrations of albumin in separate runs, whereby the binding capacity could be established in the form of albumin-to-substance ratios.

For these examinations, a Biosep-SEC-s 4000 column, 300×4.6 mm micron (Phenomenenx) on a Shimadzu HPLC installation was used. The buffer flow rate was 0.35 ml/min, the column having been equilibrated with 50 mM of potassium phosphate buffer, pH 7.4. The protein concentration was 50 μM, and the injection volume was 80 μl. Phenylbutazone was monitored at 263 nm, and warfarin at 308 nm. The regions of linear absorption had been determined beforehand.

The albumin as described in this application (1) as well as two commercially obtainable (stabilized) albumin preparations (2, 3) were used. They were 20% albumin solutions.

FIG. 1 shows a superposition of four different chromatograms, the column having been equilibrated in 50 μM phenylbutazone (in phosphate buffer). At a retention time of 11 minutes, the albumin became eluted first, the peak indicating the sum of protein absorption and that of the bound substance. At 14.5 min, an N-acetyltryptophan (stabilizer) peak is usually found in the case of a commercial albumin. After 18.5 min, the “vacancy” peak appears in the form of a “negative” representation of the absorption relative to the level of the equilibration buffer including the substance. The higher (in a negative sense) this peak or the larger the peak area, the more substance has bound to the previously eluted albumin.

FIG. 2 shows the ultraviolet absorptions of three concentrations of phenylbutazone bound to albumin (after subtraction of the buffer peak). Thus, two commercially available albumins (containing caprylate and N-acetyltryptophan) and the albumin prepared by the process described in the present application were subjected to chromatography, and the binding qualities compared. For comparable molar concentrations of phenylbutazone to albumin, it is clearly found that the peaks are significantly larger in terms of height and area in the case of the novel albumin. This similarly holds for the second example, namely warfarin, as shown in FIG. 3.

These results underscore that the commercial albumin is inferior to the albumin described herein with respect to binding property.

Comparison of the Binding Capacity of RP-18 Columns as Compared to Polystyrene-divinylbenzene Polymers (Amberchrome 161 M).

Test system: column volume: 44 ml

Flow rate: 4 ml/min

The column was charged with 1% Triton X-100 solution. The Triton X content in the eluate was measured after each column volume by means of reverse-phase HPLC. If Triton could be detected in the eluate, the capacity of the gel was exhausted.

Result:

The RP-18 gel binds 140 mg of Triton X-100/ml of gel, and the Amberchrome gel binds 160 mg of Triton X-100/ml of gel.

1. A process for the preparation of albumin, comprising the following steps:
(a) subjecting a first aqueous albumin solution to a treatment for virus inactivation by the SD method by contacting it with SD reagents at a temperature of below 45° C.;
(b) removing, at least substantially, the SD reagents by oil extraction followed by hydrophobic interaction chromatography, wherein a hydrophobic matrix, is used for said chromatography, with the proviso that said groups are aliphatic groups with more than 24 carbon atoms, to obtain a second albumin solution;
(c) optionally adding one or more stabilizers selected from the group consisting of sugars, amino acids and sugar alcohols, with the proviso that no indole stabilizer and no C₆-C₁₀ fatty acid is employed as said stabilizer, and
(d) subjecting said second albumin solution to which a stabilizer has optionally been added is subjected to final packaging and sterile filtration and optionally filled into final containers.

2. The process according to claim 1, wherein said virus inactivation is effected at a temperature within a range of from 25 to 40° C.

3. The process according to claim 1, wherein said virus inactivation is effected during a period of time within a range of from 4 to 6 hours.

4. The process according to claim 1, wherein said stabilizer is glycine, glutamate, arginine or lysine or a combination thereof.

5. The process according to claim 1, wherein said stabilizer is maltose and/or sorbitol.

6. The process according to claim 1, wherein castor oil is employed for oil extraction.

7. The process according to claim 1, wherein said hydrophobic matrix is a polystyrene-divinylbenzene polymer or a methacrylate-based polymer.

8. The process according to claim 1, wherein branched or linear aliphatic groups with more than 24 carbon atoms are bound to said matrix.

9. The process according to claim 1, wherein the albumin solution is deep-frozen after being filled into final containers.

10. The process according to claim 1, wherein any prekallikrein activator (PKA) activity which may be present before or after steps (a), (b) or (c) is removed.

11. The process according to claim 10, wherein said albumin solution is
(a) contacted with active charcoal, followed by removing the active charcoal from the albumin solution; or
(b) subjected to ion-exchange chromatography;
to remove any prekallikrein activator activity which may be present.

12. The process according to claim 11, wherein step a) is performed at an albumin concentration of from 1 to 25% by weight.

13. The process according to claim 12, wherein said albumin concentration is from 5 to 10% by weight.

14. The process according to claim 11, wherein step b) is performed at an albumin concentration of from 5 to 10% by weight.

15. The process according to claim 11, wherein said ion-exchanger is an anion exchanger, the albumin solution is buffered with sodium acetate within a range of from 100 to 150 mmol/l, and the pH is within a range of from 5.0 to 6.0.

16. The process according to claim 15, wherein the pH is less than 5.5.

17. The process according to claim 11, wherein said ion-exchanger is a cation exchanger, the albumin solution is buffered with sodium acetate within a range of from 20 to 30 mmol/l, and the pH value is within a range of from 4.8 to 6.0.

18. The process according to claim 17, wherein the pH is within a range of from 4.8 to 5.2.

19. An albumin solution obtainable by the process according to claim 1.
